(19) United States
(12) Patent Application Publication
(10) Pub. No.: US 2025/0032527 A1
(43) Pub. Date: Jan. 30, 2025

(54) MEDICAL USE OF N4-HYDROXY CITICOLINE COMPOUNDS

(71) Applicant: IMMUNIC AG, Gräfelfing (DE)

(72) Inventors: Christian GEGE, Ehingen (DE); Hella KOHLHOF, Munich (DE); Andreas MÜHLER, Munich (DE); Daniel VITT, Germering (DE)

(21) Appl. No.: 18/705,970

(22) PCT Filed: Nov. 1, 2022
(86) PCT No.: PCT/EP2022/080430

§ 371 Date: Apr. 29, 2024

(30) Foreign Application Priority Data
Nov. 1, 2021 (EP) 21205795.4

(51) Int. Cl. A61K 31/7068; A61K 31/196; A61P 31/14; A61P 31/16; C07H 19/10

(52) U.S. Cl. CPC A61K 31/7068; A61K 31/196; A61P 31/14; A61P 31/16; C07H 19/10

(57) ABSTRACT

The present disclosure relates to the medical use of citicoline analogs with a N⁴-hydroxycytidine moiety and a solvate or pharmaceutically acceptable salt thereof. The compounds can be used as antiviral drug for treatment of e.g. Covid-19, human rhinovirus (HRV), influenza and respiratory syncytial virus (RSV).

### SUMMARY OF THE INVENTION

The present disclosure relates to the medical use of citicoline analogs with a N⁴-hydroxycytidine moiety. These analogs can be used as antiviral drugs for treatment of e.g. Covid-19, human rhinovirus (HRV), influenza and respiratory syncytial virus (RSV).

### BACKGROUND OF THE INVENTION

Citicoline, also known as cytidine diphosphate-choline (CDP-choline) or cytidine 5′-diphosphocholine is an intermediate in the generation of phosphatidylcholine from choline, a common biochemical process in cell membranes. Citicoline is naturally occurring in the cells of human and animal tissue, in particular the organs. Citicoline is commercially available as dietary supplement. When taken as a dietary supplement, citicoline is hydrolyzed into choline and cytidine in the intestine. Once these fragments (i.e. choline, cytidine) cross the blood-brain barrier it is reformed into citicoline by the rate-limiting enzyme in phosphatidylcholine synthesis, CTP-phosphocholine cytidylyltransferase. Citicoline is water-soluble, with more than 90% oral bioavailability and has a very low toxicity profile in animals and humans. Citicoline as the active ingredient is, for example, described as a preventive/remedy for drug-induced neuropathy in WO2004/006940. These advantageous overall properties make citicoline an excellent starting point for drug development. However, no antiviral activity has been mentioned for citicoline. By replacing a hydrogen with a hydroxy group in the 4-position of the cytosine moiety, the resulting compound showed surprisingly antiviral activity against SARS-COV-2, which can be even more improved in a synergistic behavior, when combined with an DHODH inhibitor.

Cytidine can be chemically hydroxylated at the nitrogen in 4-position of the cytosine to form β-D-N⁴-hydroxycytidine (NHC), also known as EIDD-1931. The isobutyric acid in the 5′-position of the ribose is an prodrug of EIDD-1931, named molnupiravir or EIDD-2801. Both compounds are antiviral drugs exerting their antiviral action through introduction of copying errors during viral RNA replication (Nat. Struct. Mol. Biol. 2021; 28:740). Molnupiravir is currently developed for the treatment of influenza and approved for the treatment of Covid-19. When administered orally, molnupiravir is hydrolyzed to NHC, which is subsequently metabolized to β-D-N⁴-hydroxycytidine 5′-triphosphate (EIDD-2061). During replication, the virus's RNA polymerase enzyme incorporates EIDD-2061 into newly made RNA instead of using real cytidine.

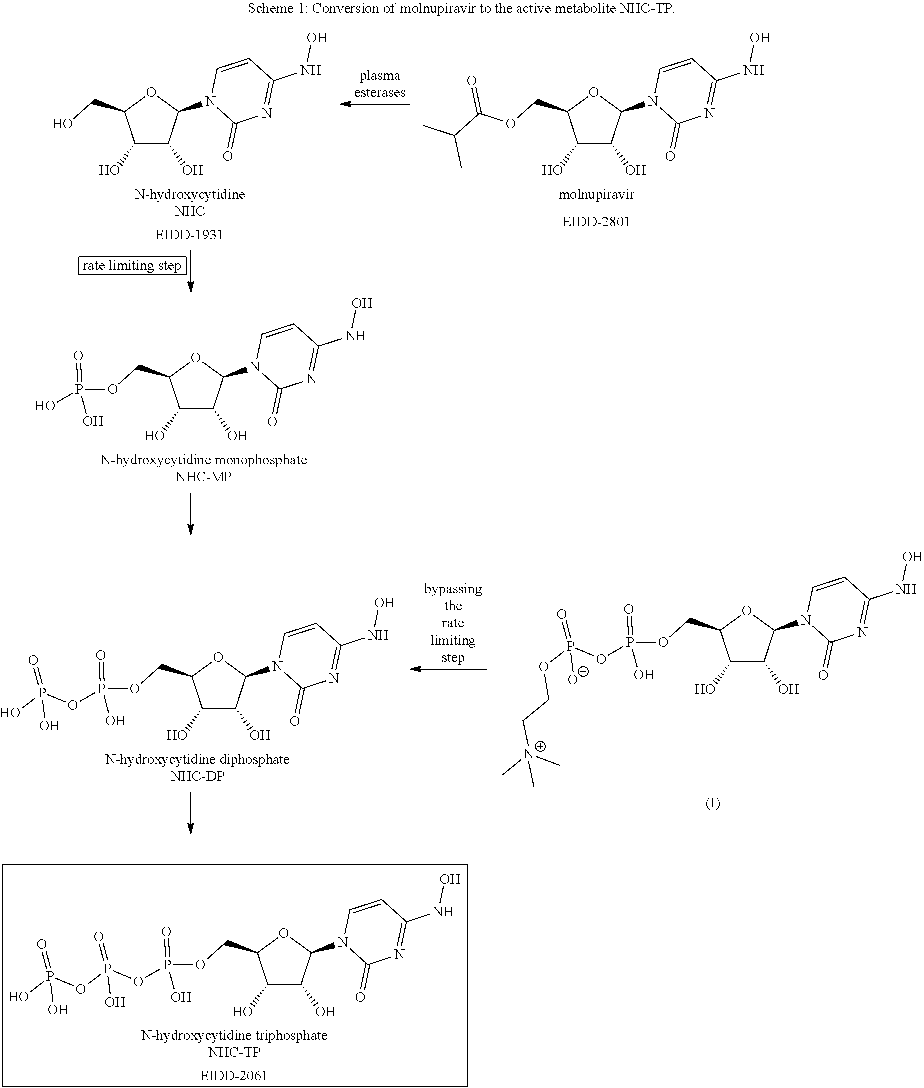

The disadvantage of molnupiravir is that the prodrug must first be hydrolysed to β-D-NA-hydroxycytidine, which however is not active in the human body but requires intracellular activation by host cell kinases. These enzymes convert the nucleoside analog via the mono-and diphosphate to the ultimately active nucleoside analog triphosphate. The conversion to the monophosphate is the rate-limiting step in this process.

In the present invention, we improve bioactivation by using the diphosphate-choline prodrug (Formula (I)), in which the β-D-N⁴-hydroxycytidine 5′-diphosphate is formed after cleavage of choline, thus bypassing the rate-limiting step. In addition, we show surprising effects for the nucleoside analog of Formula (I) compared to molnupiravir and NHC, e.g. improved permeability despite low solubility (Example 3) and high concentrations of NHC-DP and NHC-TP in plasma over a long period of time (Example 4). In addition, we describe a straightforward one-step synthesis of a compound according to Formula (I) from readily available and cheap cytidine diphosphate-choline (CDP-choline or citicoline), which is sold as dietary supplement in pharmaceutical grade quality (Example 1 and Example 2).

### PRIOR ART

The compound of the present invention according to Formula (I) is the N⁴-hydroxy analog of citicoline an be found in SciFinder with CAS-number 13186-92-0 and 13186-58-8 and no other salts are described except the depicted zwitterion.

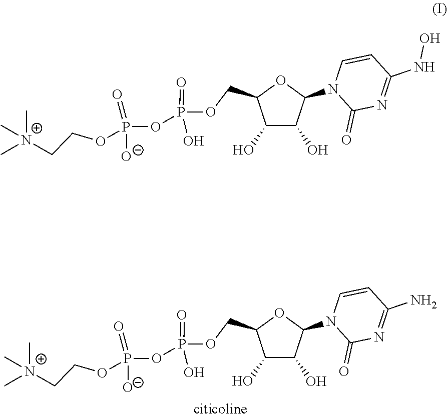 [SMILES: C[N+](C)(C)CCOP(=O)([O-])OP(=O)(O)OC[C@H]1O[C@@H](n2ccc(NO)nc2=O)[C@H](O)[C@@H]1O ; C[N+](C)(C)CCOP(=O)([O-])OP(=O)(O)OC[C@H]1O[C@@H](n2ccc(N)nc2=O)[C@H](O)[C@@H]1O]

The only linked reference towards Formula (I) is a publication from Abdelrahman et al. (J. Sep. Sci. 2020; 43:2981), which describes the formation of this compound upon oxidative degradation of citicoline (using 3% hydrogen peroxide) and its use as reference standard in the analytical development and validation of stability-indicating chromatographic methods for simultaneous determination of citicoline and piracetam.

WO2019/113462 (Emory University) describes the preparation of N⁴-hydroxycytidine and derivatives and antiviral uses related thereto. The application describes many different ester prodrugs of N⁴-hydroxycytidine including molnupiravir.

Additional patent applications from Emory University WO2017/156380 and WO2016/106050 also describe the reparation of N⁴-hydroxycytidine prodrugs and antiviral uses related thereto. The 5′-O-position of the ribose moiety can also be substituted with a broad range of residues R¹, e.g. monophosphate, diphosphate, triphosphate which can be optionally substituted with one or more residues R²⁰. This R²⁰ mentions a lot of groups including alkyl.

The free acid of β-D-N⁴-hydroxycytidine 5′-triphosphate (NHC-TP, CAS number 34973-27-8) and β-D-N⁴-hydroxycytidine 5′-diphosphate (NHC-DP, CAS number 39023-73-9) are commercially available, e.g. from MedChemExpress.

Several former patent applications, e.g. WO2002/100415 (Roche) or WO2016/100569 (Alios) mention the diphosphate moiety, which however can't be substituted to yield an ester.

### BRIEF DESCRIPTION OF THE DRAWING

FIG. 1 depicts a representative result of an experiment wherein Example 1 is combined with an DHODH inhibitor. The data shows a synergistic antiviral effect on SARS-COV-2 at different doses.

FIG. 2 depicts the blood levels of NHC and its phosphorylated derivatives after dosage of Example 2 in mice.

### SUMMARY OF THE INVENTION

The present disclosure relates to the medical use of citicoline analogs having a N⁴-hydroxycytidine moiety. These analogs can be used as antiviral drugs for treatment of e.g. Covid-19, human rhinovirus (HRV), influenza and respiratory syncytial virus (RSV).

### DETAILED DESCRIPTION OF THE INVENTION

Subject matter of the present invention is described by the following embodiments:

1. A compound according to Formula (I):

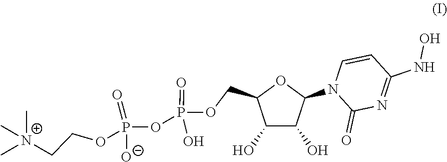 [SMILES: C[N+](C)(C)CCOP(=O)([O-])OP(=O)(O)OC[C@H]1O[C@@H](n2ccc(NO)nc2=O)[C@H](O)[C@@H]1O]

or a tautomer, solvate or pharmaceutically acceptable salt thereof for use as a medicament.

2. The compound of Formula (I) or a tautomer, solvate or pharmaceutically acceptable salt thereof for use as a medicament according to embodiment 1, in the prevention and/or treatment of a disease caused by viral infection in a mammalian subject, wherein said viral infection is caused by an RNA virus, such as, for example HIV, HCV, Ebola, rotavirus, Zika virus, polio virus, rhinovirus, hepatitis A virus, measles virus, mumps virus, RSV, rabies, Lassa virus, hantavirus, or influenza, in particular a single stranded RNA virus, such as HCoV-229E, HCoV-NL63, or betacoronaviruses, such as HCoV-OC43, SARS-COV-1, HCoV-HKU1, MERS-CoV or SARS-COV-2.

3. The compound of Formula (I) or a tautomer, solvate or pharmaceutically acceptable salt thereof for use as a medicament according to embodiment 1 or 2, in the prevention and/or treatment of a disease caused by viral infection with coronaviruses in a human.

4. The compound of Formula (I) or a tautomer, solvate or pharmaceutically acceptable salt thereof for use as a medicament according to any of embodiments 1 to 3, in the prevention and/or treatment of a disease, wherein said disease is selected from AIDS, hepatitis, ebola, polio, diarrhea, measles, mumps, rabies, Lassa fever, viral flu, respiratory disease, acute respiratory disease, sepsis, acute respiratory distress syndrome, long COVID, and adverse immune reactions, in particular moderate to severe cases of said diseases, and wherein said disease preferably is selected from diseases causes by SARS-COV-2, in particular COVID-19, such as acute respiratory disease, sepsis, acute respiratory distress syndrome, long COVID and adverse immune reactions, such as a cytokine storm.

5. The compound of Formula (I) or a tautomer, solvate or pharmaceutically acceptable salt thereof for use as a medicament according to any of embodiments 1 to 4, in the prevention and/or treatment of a disease, wherein said prevention and/or treatment is in combination with a standard antiviral therapy (SAT).

6. The compound of Formula (I) or a tautomer, solvate or pharmaceutically acceptable salt thereof for use as a medicament of a compound according to any of embodiments 1 to 4, in the prevention and/or treatment of a disease, wherein said prevention and/or treatment is in combination with an DHODH inhibitor, and optionally with a standard antiviral therapy (SAT).

7. The compound of Formula (I) or a tautomer, solvate or pharmaceutically acceptable salt thereof for use as a medicament according to embodiment 6, wherein in said combination the DHODH inhibitor consists of Formula (II):

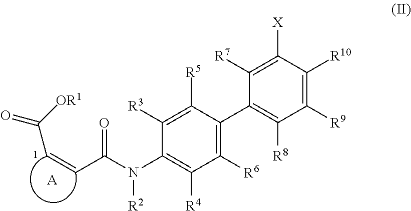

or an enantiomer, diastereomer, tautomer, prodrug, solvate, or pharmaceutically acceptable salt thereof, wherein
R¹ and R² are independently selected from H and D;
R³, R⁴, R⁵ and R⁶ are independently selected from H, D, halogen, CN, C₁₋₄-alkyl, O-C₁₋₄-alkyl, fluoro-C₁₋₄-alkyl and O-fluoro-C₁₋₄-alkyl, having one or more hydrogen atoms in alkyl optionally replaced by deuterium;
R⁷, R⁸, R⁹ and R¹⁰ are independently selected from H, D, halogen, CN, C₁₋₄-alkyl, O-C₁₋₄-alkyl, fluoro-C₁₋₄-alkyl and O-fluoro-C₁₋₄-alkyl, having one or more hydrogen atoms in alkyl optionally replaced by deuterium;
X is selected from H, D, OH, OD, S(═O)_{y}R¹¹ and OR^{11;}
R¹¹ is selected from C₁₋₄-alkyl, C₃-4-cycloalkyl and fluoro-C₁₋₄-alkyl, having one or more hydrogen atoms in alkyl optionally replaced by deuterium;
y is 0 to 2;

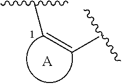

is selected from a 5-membered heteroaryl, cyclopentenyl and heterocyclopentenyl, having one or more hydrogen atoms optionally replaced by deuterium, said A is unsubstituted or substituted with 1 to 5 substituents independently selected from the group consisting of halogen, CN, NO₂, oxo, OH, C₁₋₄-alkyl, O-C₁₋₄-alkyl, fluoro-C₁₋₄-alkyl and O-fluoro-C₁₋₄-alkyl, CO₂H and SO₃H, having one or more hydrogen atoms in alkyl optionally replaced by deuterium;
provided that at least one hydrogen in R³, R⁴, R⁵, R⁶, R⁷, R⁸, R⁹, R¹⁰, R¹¹, ×and/or A is replaced by deuterium.

8. The compound of Formula (I) or a tautomer, solvate or pharmaceutically acceptable salt thereof for use as a medicament according to embodiment 7, wherein in said combination the DHODH inhibitor is selected from

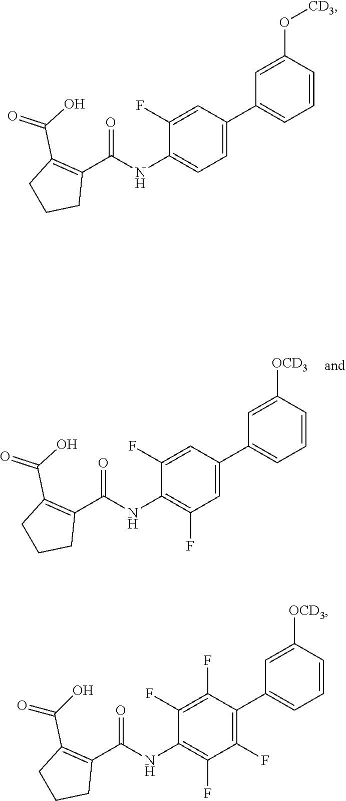

or a solvate or pharmaceutically acceptable salt thereof.

9. A pharmaceutical composition comprising the compound of Formula (I) or a tautomer, solvate or pharmaceutically acceptable salt thereof for use as a medicament according to any of embodiments 1 to 8, such as a tablet, capsule, granule, powder, sachet, reconstitutable powder, dry powder inhaler and/or chewable.

10. A pharmaceutical composition according to embodiment 9, wherein said pharmaceutical composition further comprises one or more pharmaceutically acceptable carriers or excipients.

11. A compound according to Formula (I)

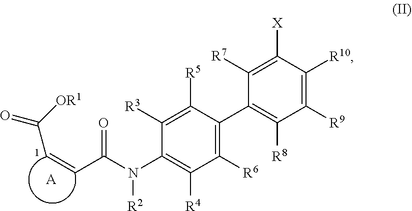

which is obtainable by reacting citicoline or salts thereof with hydroxylamine or salts thereof.

12. A compound according to Formula (I):

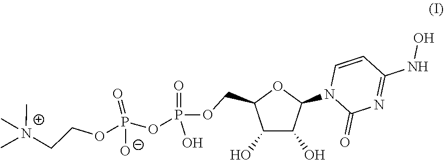

or a tautomer, solvate or pharmaceutically acceptable salt thereof for use in the treatment of a disease selected from AIDS, hepatitis, ebola, polio, diarrhea, measles, mumps, rabies, Lassa fever, viral flu, respiratory disease, acute respiratory disease, sepsis, acute respiratory distress syndrome, long COVID, and adverse immune reactions, in particular moderate to severe cases of said diseases, and wherein said disease preferably is selected from diseases causes by SARS-COV-2, in particular COVID-19, such as acute respiratory disease, sepsis, acute respiratory distress syndrome, long COVID and adverse immune reactions, such as a cytokine storm.

13. Use of a compound according to Formula (I):

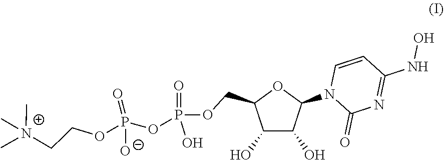

or a tautomer, solvate or pharmaceutically acceptable salt thereof as a medicament.

14. The use of a compound according to Formula (I) or a tautomer, solvate or pharmaceutically acceptable salt thereof as a medicament according to embodiment 13, in the prevention and/or treatment of a disease caused by viral infection in a mammalian subject, wherein said viral infection is caused by an RNA virus, such as, for example HIV, HCV, Ebola, rotavirus, Zika virus, polio virus, rhinovirus, hepatitis A virus, measles virus, mumps virus, RSV, rabies, Lassa virus, hantavirus, or influenza, in particular a single stranded RNA virus, such as HCoV-229E, HCoV-NL63, or betacoronaviruses, such as HCoV-OC43, SARS-COV-1, HCoV-HKU1, MERS-CoV or SARS-COV-2.

15. The use of a compound according to Formula (I) or a tautomer, solvate or pharmaceutically acceptable salt thereof as a medicament according to embodiment 13 or 14, in the prevention and/or treatment of a disease caused by viral infection with coronaviruses in a human.

16. The use of a compound according to Formula (I) or a tautomer, solvate or pharmaceutically acceptable salt thereof as a medicament according to any of embodiments 13 to 15, in the prevention and/or treatment of a disease, wherein said disease is selected from AIDS, hepatitis, ebola, polio, diarrhea, measles, mumps, rabies, Lassa fever, viral flu, respiratory disease, acute respiratory disease, sepsis, acute respiratory distress syndrome, long COVID, and adverse immune reactions, in particular moderate to severe cases of said diseases, and wherein said disease preferably is selected from diseases causes by SARS-COV-2, in particular COVID-19, such as acute respiratory disease, sepsis, acute respiratory distress syndrome, long COVID and adverse immune reactions, such as a cytokine storm.

17. The use of a compound according to Formula (I) or a tautomer, solvate or pharmaceutically acceptable salt thereof as a medicament according to any of embodiments 13 to 16, in the prevention and/or treatment of a disease, wherein said prevention and/or treatment is in combination with a standard antiviral therapy (SAT).

18. The use of a compound according to Formula (I) or a tautomer, solvate or pharmaceutically acceptable salt thereof as a medicament according to any of embodiments 13 to 16, in the prevention and/or treatment of a disease, wherein said prevention and/or treatment is in combination with an DHODH inhibitor, and optionally with a standard antiviral therapy (SAT).

19. The use of a compound according to Formula (I) or a tautomer, solvate or pharmaceutically acceptable salt thereof as a medicament according to embodiment 18, wherein in said combination the DHODH inhibitor consists of Formula (II):

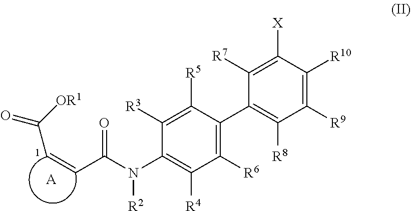

or an enantiomer, diastereomer, tautomer, prodrug, solvate, or pharmaceutically acceptable salt thereof, wherein

R¹ and R² are independently selected from H and D;
R³, R⁴, R⁵ and R⁶ are independently selected from H, D, halogen, CN, C₁₋₄-alkyl, O-C₁₋₄-alkyl, fluoro-C₁₋₄-alkyl and O-fluoro-C₁₋₄-alkyl, having one or more hydrogen atoms in alkyl optionally replaced by deuterium;
R⁷, R⁸, R⁹ and R¹⁰ are independently selected from H, D, halogen, CN, C₁₋₄-alkyl, O-C₁₋₄-alkyl, fluoro-C₁₋₄-alkyl and O-fluoro-C₁₋₄-alkyl, having one or more hydrogen atoms in alkyl optionally replaced by deuterium;
X is selected from H, D, OH, OD, S(═O)_{y}R¹¹ and OR^{11;}
R¹¹ is selected from C14-alkyl, C34-cycloalkyl and fluoro-C14-alkyl, having one or more hydrogen atoms in alkyl optionally replaced by deuterium;
y is 0 to 2;

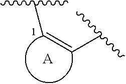

is selected from a 5-membered heteroaryl, cyclopentenyl and heterocyclopentenyl, having one or more hydrogen atoms optionally replaced by deuterium,
said A is unsubstituted or substituted with 1 to 5 substituents independently selected from the group consisting of halogen, CN, NO₂, oxo, OH, C₁₋₄-alkyl, O-C₁₋₄-alkyl, fluoro-C₁₋₄-alkyl and O-fluoro-C₁₋₄-alkyl, CO₂H and SO₃H, having one or more hydrogen atoms in alkyl optionally replaced by deuterium;
provided that at least one hydrogen in R³, R⁴, R⁵, R⁶, R⁷, R⁸, R⁹, R¹⁰, R¹¹, X and/or A is replaced by deuterium.

20. The use of a compound according to Formula (I) or a tautomer, solvate or pharmaceutically acceptable salt thereof as a medicament according to embodiment 19, wherein in said combination the DHODH inhibitor is selected from

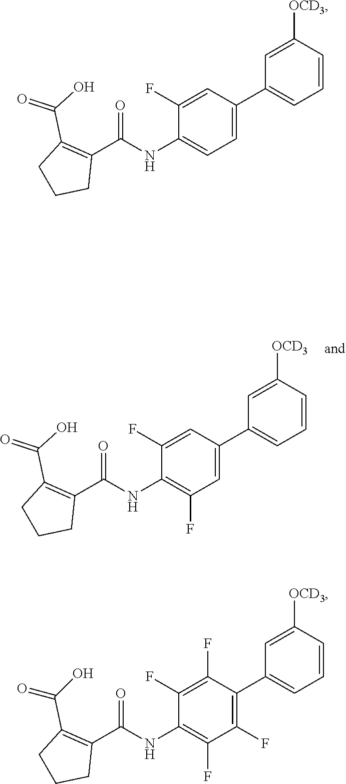

or a solvate or pharmaceutically acceptable salt thereof.

21. The use of a compound according to Formula (I) or a tautomer, solvate or pharmaceutically acceptable salt thereof as a medicament according to embodiment 13 to 20, wherein said compound(s) is/are provided as a suitable pharmaceutical composition, such as a tablet, capsule, granule, powder, sachet, reconstitutable powder, dry powder inhaler and/or chewable.

22. The use of a compound according to Formula (I) or a tautomer, solvate or pharmaceutically acceptable salt thereof as a medicament according to embodiment 21, wherein said pharmaceutical composition further include one or more pharmaceutically acceptable carriers or excipients.

23. The use of a compound according to Formula (I):

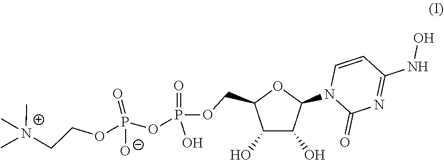

or a tautomer, solvate or pharmaceutically acceptable salt thereof in the treatment of a disease selected from AIDS, hepatitis, ebola, polio, diarrhea, measles, mumps, rabies, Lassa fever, viral flu, respiratory disease, acute respiratory disease, sepsis, acute respiratory distress syndrome, long COVID, and adverse immune reactions, in particular moderate to severe cases of said diseases, and wherein said disease preferably is selected from diseases causes by SARS-COV-2, in particular COVID-19, such as acute respiratory disease, sepsis, acute respiratory distress syndrome, long COVID and adverse immune reactions, such as a cytokine storm.

24. A method of treating or prevention a disease caused by viral infection in a mammalian subject, wherein said viral infection is caused by an RNA virus, such as, for example HIV, HCV, Ebola, rotavirus, Zika virus, polio virus, rhinovirus, hepatitis A virus, measles virus, mumps virus, RSV, rabies, Lassa virus, hantavirus, or influenza, in particular a single stranded RNA virus, such as HCoV-229E, HCoV-NL63, or betacoronaviruses, such as HCoV-OC43, SARS-CoV-1, HCoV-HKU1, MERS-COV or SARS-COV-2 comprising administering to a patient in need of such treatment or prevention a therapeutically or prophylactically effective amount of a compound according to Formula (I):

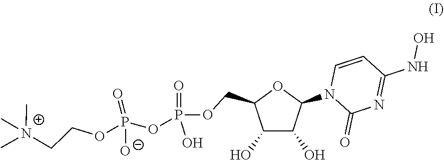

or a tautomer, solvate or pharmaceutically acceptable salt thereof.

25. The method according to embodiment 24, wherein said disease caused by viral infection with coronaviruses in a human.

26. The method according to embodiment 24 or 25, wherein said disease is selected from AIDS, hepatitis, ebola, polio, diarrhea, measles, mumps, rabies, Lassa fever, viral flu, respiratory disease, acute respiratory disease, sepsis, acute respiratory distress syndrome, long COVID, and adverse immune reactions, in particular moderate to severe cases of said diseases, and wherein said disease preferably is selected from diseases causes by SARS-CoV-2, in particular COVID-19, such as acute respiratory disease, sepsis, acute respiratory distress syndrome, long COVID and adverse immune reactions, such as a cytokine storm.

27. The method according to any of embodiments 24 to 26, wherein said prevention and/or treatment is in combination with a standard antiviral therapy (SAT).

28. The method according to any of embodiments 24 to 26, wherein said prevention and/or treatment is in combination with an DHODH inhibitor, and optionally with a standard antiviral therapy (SAT).

29. The method according to embodiment 28, wherein in said combination the DHODH inhibitor consists of Formula (II):

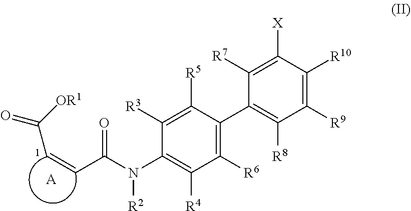

or an enantiomer, diastereomer, tautomer, prodrug, solvate, or pharmaceutically acceptable salt thereof, wherein
R¹ and R² are independently selected from H and D;
R³, R⁴, R⁵ and R⁶ are independently selected from H, D, halogen, CN, C₁₋₄-alkyl, O-C₁₋₄-alkyl, fluoro-C₁₋₄-alkyl and O-fluoro-C₁₋₄-alkyl, having one or more hydrogen atoms in alkyl optionally replaced by deuterium;
R⁷, R⁸, R⁹ and R¹⁰ are independently selected from H, D, halogen, CN, C₁₋₄-alkyl, O-C₁₋₄-alkyl, fluoro-C₁₋₄-alkyl and O-fluoro-C₁₋₄-alkyl, having one or more hydrogen atoms in alkyl optionally replaced by deuterium;
X is selected from H, D, OH, OD, S(═O)_{y}R¹¹ and OR^{11;}
R¹¹ is selected from C14-alkyl, C34-cycloalkyl and fluoro-C14-alkyl, having one or more hydrogen atoms in alkyl optionally replaced by deuterium;
y is 0 to 2;

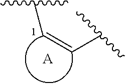

is selected from a 5-membered heteroaryl, cyclopentenyl and heterocyclopentenyl, having one or more hydrogen atoms optionally replaced by deuterium,
said A is unsubstituted or substituted with 1 to 5 substituents independently selected from the group consisting of halogen, CN, NO₂, oxo, OH, C₁₋₄-alkyl, O-C₁₋₄-alkyl, fluoro-C₁₋₄-alkyl and O-fluoro-C14-alkyl, CO₂H and SO₃H, having one or more hydrogen atoms in alkyl optionally replaced by deuterium;
provided that at least one hydrogen in R³, R⁴, R⁵, R⁶, R⁷, R⁸, R⁹, R¹⁰, R¹¹, X and/or A is replaced by deuterium.

30. The method according to embodiment 29, wherein in said combination the DHODH inhibitor is selected from

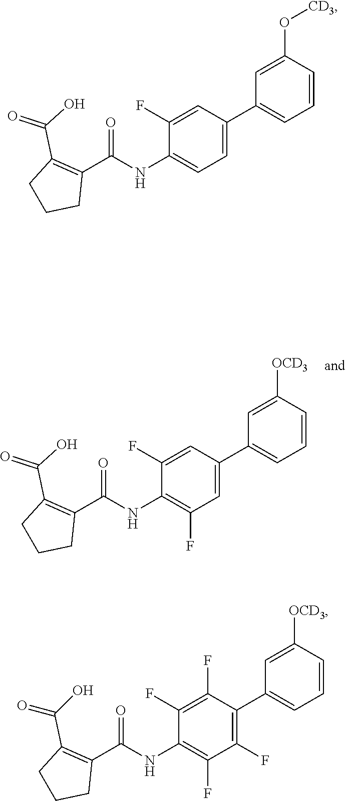

or a solvate or pharmaceutically acceptable salt thereof.

31. A method of treatment of disease selected from AIDS, hepatitis, ebola, polio, diarrhea, measles, mumps, rabies, Lassa fever, viral flu, respiratory disease, acute respiratory disease, sepsis, acute respiratory distress syndrome, long COVID, and adverse immune reactions, in particular moderate to severe cases of said diseases, and wherein said disease preferably is selected from diseases causes by SARS-COV-2, in particular COVID-19, such as acute respiratory disease, sepsis, acute respiratory distress syndrome, long COVID and adverse immune reactions, such as a cytokine storm comprising administering to a patient in need of such treatment or prevention a therapeutically or prophylactically effective amount of a compound according to Formula (I):

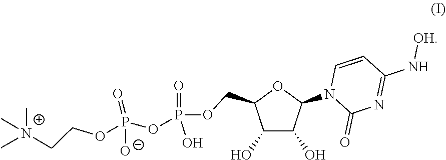

The invention further relates to the use of a compound according to Formula (I):

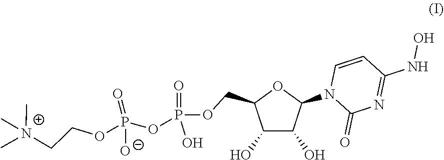

or a tautomer, solvate or pharmaceutically acceptable salt thereof in the manufacture of a medicament as defined in any of the above embodiments.

### DEFINITIONS AND PARTICULAR EMBODIMENTS

An DHODH inhibitor for combination with a compound according to Formula (I) is selected from vidofludimus, vidofludimus calcium (IMU-838), teriflunomide, leflunomide, emvododstat (PTC299), brequinar, farudostat (ASLAN003), PP-001, RP7214, orludodstat (BAY2402234), JNJ-74856665, AG-636, MEDS433, or a compound according to Formula (II), which have been described in provisional application EP21167690. Preferably, the DHODH inhibitor is a compound according to Formula (II). More preferably, the DHODH inhibitor is selected from

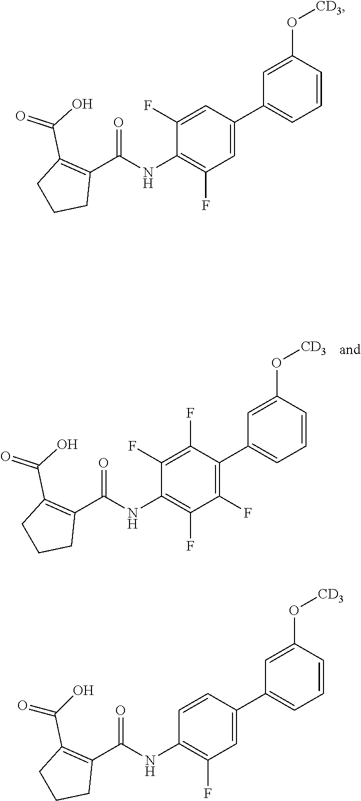 [SMILES: [2H]C([2H])([2H])Oc1cccc(-c2cc(F)c(NC(=O)C3=C(C(=O)O)CCC3)c(F)c2)c1 ; [2H]C([2H])([2H])Oc1cccc(-c2c(F)c(F)c(NC(=O)C3=C(C(=O)O)CCC3)c(F)c2F)c1 ; [2H]C([2H])([2H])Oc1cccc(-c2ccc(NC(=O)C3=C(C(=O)O)CCC3)c(F)c2)c1]

or a solvate
or pharmaceutically acceptable salt thereof.

Examples of other therapeutic agents used in a standard antiviral therapy (SAT) that may be combined with the compound of Formula (I), either administered separately, co-administered or in the same pharmaceutical composition include, but are not limited to a:
(1) Protease inhibitor (e.g. PF-07304814, PF-00835231, nirmatrelvir, lopinavir or ritonavir);
(2) Polymerase inhibitor (e.g. gemcitabine);
(3) Allosteric polymerase inhibitor;
(4) Interferon alfa-2a, which may be pegylated or otherwise modified, and/or ribavirin;
(5) Non-substrate-based inhibitor;
(6) Helicase inhibitor;
(7) Primase-helicase inhibitor (e.g. pritelivir);
(8) Antisense oligodeoxynucleotide (S-ODN);
(9) Aptamer;
(10) Nuclease-resistant ribozyme;
(11) IRNA, including microRNA and SiRNA;
(12) Antibody, partial antibody or domain antibody to the virus;
(13) Viral antigen or partial antigen that induces a host antibody response;
(14) NOD-, LRR-and pyrin domain-containing protein 3 (NLRP3);
(15) Anti-coronavirus vaccine therapy;
(16) RNA replication modulator; or
(17) Neuraminidase inhibitor;
(18) Glutamyl-prolyl-tRNA synthetase inhibitor (e.g. halofuginone);
(19) Equilibrative nucleoside transporter (ENT) inhibitor (e.g. dipyridamole); or
(20) other nucleoside analogue/RNA replication modulator (e.g. molnupiravir, bemnifosbuvir).

Any formula or structure given herein, is also intended to represent isotopically labelled atoms. Examples of isotopes that can be incorporated into compounds of the disclosure include further isotopes of hydrogen (i.e. deuterium or tritium), as well as isotopes of carbon, nitrogen, oxygen, phosphorous, fluorine and chlorine, such as, but not limited to ¹¹C, ¹³C, ¹⁴C, ¹⁵N, ¹⁸F, ³¹P, ³²P, ³⁵S, ³⁶Cl and ¹²⁵I. The disclosure further comprises various isotopically labelled compounds into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or radioactive treatment of patients.

The compounds of the present invention are partly subject to tautomerism. For example, if a heteroaromatic group containing a nitrogen atom in the ring is substituted with a hydroxy or amino group on the carbon atom adjacent to the nitrogen atom, the following tautomerism can appear:

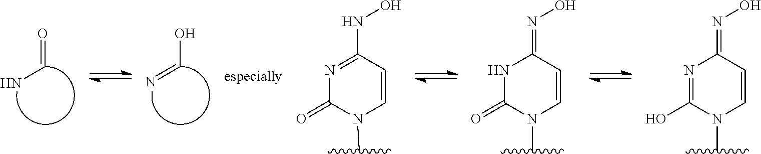

The term “pharmaceutically acceptable salts” refers to salts prepared from pharmaceutically acceptable non-toxic bases, including inorganic bases and organic bases. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The respective salts can be obtained by customary methods which are known to the person skilled in the art like, for example, by contacting these with an organic or inorganic base in a solvent or dispersant, or by cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts.

Further the compounds of the present disclosure may be present in the form of solvates, such as those which include as solvate water, or pharmaceutically acceptable solvates, such as alcohols, in particular ethanol. A stoichiometric or non-stoichiometric amount of solvent is bound by non-covalent intermolecular forces. When the solvent is water, the “solvate” is a “hydrate”. It is understood, that a “pharmaceutically acceptable salts” can in addition optionally contain a “solvate”.

The term “effective amount” is meant to include the amount of a compound that, when administered, is sufficient to prevent development of, or alleviate to some extent, one or more of the symptoms of a disorder, disease, or condition being treated. The term “effective amount” also refers to the amount of a compound that is sufficient to elicit the biological or medical response of a cell, tissue, system, animal, or human, which is being sought by a researcher, veterinarian, medical doctor, or clinician.

As used herein, the term “subject” refers to any member of the animal kingdom including humans. In some embodiments, “subject” refers to humans, at any stage of development. In some embodiments, “subject” refers to a human patient. In some embodiments, “subject” refers to non-human animals. In some embodiments, the non-human animal is a mammal (e.g. a rodent, a mouse, a rat, a rabbit, a monkey, a dog, a cat, a sheep, cattle, a primate or a pig). In some embodiments, subjects include, but are not limited to, mammals, birds, reptiles, amphibians, fish or worms. In some embodiments, a subject may be a transgenic animal, genetically engineered animal or a clone.

As used herein, the term “treating” or “treatment” includes, but is not limited to, methods and manipulations to produce beneficial changes in a recipient's health status. The changes can be either subjective or objective and can relate to features such as symptoms or signs of the viral infection being treated. Preventing the deterioration of a recipient's status is also included by the term. Treating, as used herein, also includes administering the compound of Formula (I) alone or in combination with an additional therapeutic agent to a subject having a viral infection or is at risk of becoming a viral infection.

As used herein, the term “administering” includes activities associated with providing a patient an amount of a compound described herein, e.g. a compound of Formula (I). Administering includes providing unit dosages of compositions set forth herein to a patient in need thereof. Administering includes providing effective amounts of compounds, e.g. a compound of Formula (I), for a specified period of time, e.g. for about 1, 2, 3, 4, 5, 6, 7, 8, 9, 10, 12, 13, 14, 15, 16, 17, 18, 19, 20, 21, 22, 23, 24, 25, 26, 27, 28, 29, 30, 31 or more days, or in a specified sequence, e.g. administration a compound of Formula (I) followed by the administration of one or more antiviral drugs, or vice versa.

As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. In some instances, the co-administered compounds are administered by the same route. In other instances, the co-administered compounds are administered via different routes. For example, one compound can be administered orally, and the other compound can be administered, e.g. sequentially or simultaneously, via intravenous or intraperitoneal injection.

The compound of Formula (I) or optionally the additional therapeutic agents can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral administration. The compound of Formula (I) or optionally the additional therapeutic agents can be administered alone but is generally mixed with a pharmaceutically acceptable carrier and co-administered in the form of a tablet or capsule, liposome or as an agglomerated powder. Examples of suitable solid carriers include lactose, sucrose, gelatin and agar. Capsule or tablets can be easily formulated and can be made easy to swallow or chew; other solid forms include granules and bulk powders. Tablets may contain suitable binders, lubricants, diluents, disintegrating agents, coloring agents, flavoring agents flow-inducing agents and melting agents.

Compositions may further comprise one or more pharmaceutically acceptable additional ingredient(s) such as alum, stabilizers, antimicrobial agents, buffers, coloring agents, flavoring agents, adjuvants, and the like.

Compositions may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, acacia, gelatin, sorbitol, tragacanth, mucilage of starch and polyvinylpyrrolidone. Fillers include, but are not limited to, lactose, sugar, microcrystalline cellulose, maize starch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate). Tablets may be coated according to methods well known in the art.

Compositions may also be liquid formulations including, but not limited to, aqueous or oily suspensions, solutions, emulsions, syrups, and elixirs. The compositions may also be formulated as a dry product for constitution with water or other suitable vehicle before use, such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminium stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

As used herein the viral infection, especially acute viral infection, is selected from coronavirus infections, SARS-COV-2 (COVID-19), SARS, flu/influenza (and avian influenza), HIV/Aids, chickenpox (Varicella), cytomegalovirus, Dengue Fever, German measles (Rubella), hand-foot-mouth disease, hantavirus infections, all forms of hepatitis, Lassa fever, Marburg virus infections, measles, meningitis, MERS-COV, mumps, norovirus infections, herpes simplex virus infections, smallpox, monkeypox, rotavirus infections, Ebola virus, poliovirus infections, rhinovirus infections, parainfluenzavirus infections, RSV infections, HCMV infections and bannavirus infections. Preferred is SARS-COV-2 (COVID-19), flu/influenza, rhinovirus and respiratory syncytial virus (RSV) infections. More preferred is SARS-COV-2 (COVID-19), flu/influenza and rhinovirus infections, most preferred is SARS-COV-2 (COVID-19). It is understood, that also mutated forms of the virus (e.g. of SARS-COV-2) are covered.

A virus, especially SARS-COV-2, is constantly mutating, which many increase virulence and transmission rates. Drug-resistant variants of viruses may emerge after prolonged treatment with an antiviral agent. Drug resistance may occur by mutation of a gene that encodes for an enzyme used in viral replication. The efficacy of a drug against an RNA virus infection in certain cases can be prolonged, augmented or restored by administering the compound in combination or alternation with another and perhaps even two or three other, antiviral compounds that induce a different mutation or act through a different pathway, from that of the principal drug. A variant of a known virus can refer to a virus carrying one or more nucleotide mutations in the viral genome as compared to the known virus, for instance at least 1, 2, 3, 4, 5, 6, 7, 8, 9, 10, 15, 20, 30, 40, 60, 100, 200, 300 or even more nucleotide mutations. Mutations can refer to nucleotide deletion, insertion, or substitution. In some cases, a variant can have at most 50%, 40%, 30%, 20%, 10%, 5%, 4%, 3%, 2% or 1% of the viral genome different than the genome of a known virus.

In particular embodiments of the present invention, a compound according to Formula (I) is for use as a medicament and/or for use in therapy. In a more particular embodiment of the present invention, the therapy is to prevent and/or treat a diseases caused by viral infection in a mammalian subject. In an even more particular embodiment of the present invention, the disease is caused by an RNA virus, such as, for example HIV, HCV, Ebola, rotavirus, Zika virus, polio virus, rhinovirus, hepatitis A virus, measles virus, mumps virus, RSV, rabies, Lassa virus, hantavirus, or influenza, in particular a single stranded RNA virus, such as HCoV-229E, HCoV-NL63, or betacoronaviruses, such as HCoV-OC43, SARS-COV-1, HCoV-HKU1, MERS-CoV or SARS-COV-2. In an even more particular embodiment of the present invention, the disease is caused by SARS-COV-2, rhinovirus, RSV and influenza virus. In a most particular embodiment of the present invention, the disease is caused by SARS-COV-2.

In other particular embodiments of the present invention, a compound according to Formula (I) is for use as a medicament and/or for use in therapy. In a more particular embodiment of the present invention, the therapy is to prevent and/or treat a diseases caused by viral infection in a mammalian subject. In an even more particular embodiment of the present invention, the disease is selected from AIDS, hepatitis, ebola, polio, diarrhea, measles, mumps, rabies, Lassa fever, viral flu, respiratory disease, acute respiratory disease, sepsis, acute respiratory distress syndrome, long COVID, and adverse immune reactions, in particular moderate to severe cases of said diseases, and wherein said disease preferably is selected from diseases causes by SARS-COV-2, in particular COVID-19, such as acute respiratory disease, sepsis, acute respiratory distress syndrome, long COVID and adverse immune reactions, such as a cytokine storm. In an even more particular embodiment of the present invention, the disease is COVID-19, viral flu, respiratory disease and acute respiratory disease. In a most particular embodiment of the present invention, the disease is COVID-19.

In other particular embodiments of the present invention, a compound according to Formula (I) is for use as a medicament in combination with a standard antiviral therapy (SAT). In a more particular embodiment of the present invention, this combination is with an DHODH inhibitor, and optionally with a standard antiviral therapy (SAT). In a more particular embodiment of the present invention, the DHODH inhibitor is selected from vidofludimus, vidofludimus calcium (IMU-838), teriflunomide, leflunomide, emvododstat (PTC299), brequinar, farudostat (ASLAN003), PP-001, RP7214, orludodstat (BAY2402234), JNJ-74856665, AG-636, MEDS433. In an equally more particular embodiment of the present invention, the DHODH inhibitor consists of Formula (II).

In other particular embodiments of the present invention, a compound according to Formula (1) is prepared by reacting citicoline or salts thereof with hydroxylamine or salts thereof. In a more particular embodiment of the present invention, a compound according to Formula (I) is prepared by reacting citicoline or salts thereof with hydroxylamine free base, hydroxylamine sulfate or hydroxylamine hydrochloride. In an even more particular embodiment of the present invention, a compound according to Formula (I) is prepared by reacting citicoline or salts thereof with 1 to 6 equivalents of hydroxylamine sulfate or hydroxylamine hydrochloride.

### EXPERIMENTAL PART

The introduction of the N⁴-hydroxy moiety into a cytidine derivative has been described e.g. by Paymode et al. (Org. Process Res. Dev. 2021; 25:1822) using hydroxylamine sulfate in water at elevated temperatures or by Purohit et al. (J. Med. Chem. 2012; 55:9988) or Vasudevan et al. (Chem. Commun. 2020; 56:13363) using hydroxylammonium acetate in water at 37° C. to 40° C. at pH 5.5 to 6.0.

### Experimental Section

### Comparative Example: Citicoline Sodium (C1)

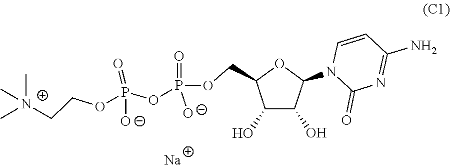 [SMILES: C[N+](C)(C)CCOP(=O)([O-])OP(=O)([O-])OC[C@H]1O[C@@H](n2ccc(N)nc2=O)[C@H](O)[C@@H]1O ; [Na+]]

Citicoline sodium (CAS: 33818-15-4) is commercially available, e.g. from MedChemExpress (Art.-Nr.: HY-B0739A) and has the following analytical data: 1H-NMR (400 MHZ, D2O) 0 7.84 (d, J=7.6 Hz, 1H), 6.01 (d, J=7.6 Hz, 1H), 5.89 (d, J=4.0 Hz, 1H), 4.30-4.06 (m, 7H), 3.59-3.57 (m, 2H), 3.12 (s, 9H). ESI-MS m/z calcd for [C₁₄H₂₇N₄O₁₁P₂⁺] [M] +: 489.1; found: 489.0.

### Example 1

2-(((((((2R,3S,4R,5R)-3,4-Dihydroxy-5-(4-(hydroxyamino)-2-oxopyrimidin-1 (2H)-yl) tetrahydrofuran-2-yl) methoxy) (hydroxy) phosphoryl) oxy) oxidophosphoryl) oxy)-N,N,N-trimethylethan-1-aminium mono sodium salt (1)

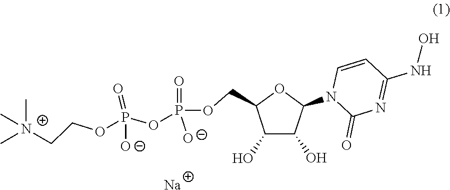 [SMILES: C[N+](C)(C)CCOP(=O)([O-])OP(=O)([O-])OC[C@H]1O[C@@H](n2ccc(NO)nc2=O)[C@H](O)[C@@H]1O ; [Na+]]

To a suspension of citicoline sodium (200 mg, 392 umol) in EtOH (5 mL) was added 5N aqueous hydroxylamine hydrochloride until the solution was adjusted to pH=5 to 6. The mixture was stirred for 8 days at 37° C. with adding 5N aqueous hydroxylamine hydrochloride intermittently to maintain the pH value. LCMS analysis indicated that most of the starting material was consumed. The solvent was removed under reduced pressure and the residue was purified by chromatography using diethylaminoethyl (DEAE) Sephadex A-25 (CAS 12609-80-2) (100 mM triethylammonium bicarbonate/H₂O gradient from 0/1 to 3/7, 20 mL/min, UV detection 254 nm). Lyophilization of the fractions gave a triethylammonium salt of the desired product. The prepared triethylammonium salt was dissolved in water and eluted through the ion-exchange column (Amberlite IR120 sodium form). Fractions containing the product were combined and lyophilized to give compound 1 (60.4 mg, 24%) as an off-white solid. 1H-NMR (400 MHZ, D2O)δ7.08 (d, J=8.4 Hz, 1H), 5.87-5.84 (m, 1H), 5.73 (d, J=8.4 Hz, 1H), 4.30-4.21 (m, 4H), 4.14-4.13 (m, 1H), 4.08-4.03 (m, 2H), 3.59-3.57 (m, 2H), 3.12 (s, 9H). ESI-MS m/z calcd for [C₁₄H₂₇N⁴⁰¹²P₂+] [M] +: 505.1; found: 505.0. Elemental analysis:

Found: C: 26.19% H: 5.99% N: 8.68%

Calc.: C: 26.14% H: 5.95% N: 8.71% for C₁₄H₂₅N₄NaO12P₂·6.5H₂O

### Example 2

2-(((((((2R,3S,4R,5R)-3,4-Dihydroxy-5-(4-(hydroxyamino)-2-oxopyrimidin-1 (2H)-yl) tetrahydrofuran-2-yl) methoxy) (hydroxy) phosphoryl) oxy) oxidophosphoryl) oxy)-N,N,N-trimethylethan-1-aminium triethylammonium salt (2)

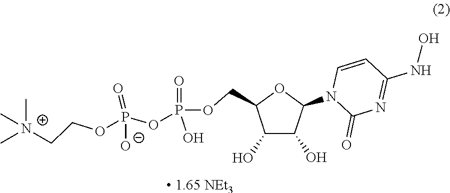 [SMILES: C[N+](C)(C)CCOP(=O)([O-])OP(=O)(O)OC[C@H]1O[C@@H](n2ccc(NO)nc2=O)[C@H](O)[C@@H]1O ; "· 1.65 NEt3"]

To a solution of citicoline (10.0 g) in H₂O (100 mL) was added hydroxylamine hydrochloride (2.8 g, 2 eq.) at 38° C. The mixture was stirred at 38° C. for 5 days. Another batch of hydroxylamine hydrochloride (2.6 g, 50% in water, 2 eq.) was added and the reaction mixture was stirred 38° C. for another 2 days. The third batch of hydroxylamine hydrochloride (2.6 g, 50% in water, 2 eq.) was added and the reaction mixture was stirred 38° C. for another 2 days. The mixture was concentrated under reduced pressure and washed with MeOH (30 mL) to give a crude product. The resulting crude was dissolved in H₂O and directly purified by prep-HPLC (column: Phenomenex Luna C₁₈ 250 mm×100 mm×10 um; mobile phase: [water (20 mM triethylammonium bicarbonate)-ACN]; B %: 0%-5%, 20 min) to get pure compound 2 (1.0 g, 7.3%, as 1.65 TEA salt) as a white solid. HPLC: 214 nm: 98.52% purity; 254 nm: 99.87% purity; 260 nm: >99.9% purity.

### Example 3: Solubility, logD and Caco-2 permeability

The aqueous solubility (in PBS, PH 7.4-cat ref: 435; in simulated intestinal fluid-cat ref: 2062 and in simulated gastric fluid-cat ref: 2061), partition coefficient (logD, n-octanol/PBS, PH 7.4; cat ref: 417) and permeability (Caco-2, pH 6.5/7.4; cat ref: 3318 and 3320) of Example 1 and comparative example (see Scheme 1) were measured at Eurofins according their standard procedures and furnished following results:

| | Client | | Wavelength | | | | | |
|---|---|---|---|---|---|---|---|---|
| Compound | Compound | Test | of | Solubility (μM) | | | Chromatographic | |
| I.D. | I.D. | Conc. | Detection | 1^{st} | 2^{nd} | Mean | Purity (%) | Flags |

Aqueous solubility (PBS, pH 7.4)

| | | | | | | | | |
|---|---|---|---|---|---|---|---|---|
| 100060338-1 | Example 1 | 2.0E−04M | 230 nm | 14.20 | 14.42 | 14.3 | 100 | |
| 100060338-2 | NHC | 2.0E−04M | 230 nm | 349.99 | 362.05 | 200 | 100 | ADJ |
| 100060338-3 | molnupiravir | 2.0E−04M | 230 nm | 246.79 | 290.52 | 200 | 100 | ADJ |
| 100060338-4 | NHC-DP | 2.0E−04M | 205 nm | — | — | — | — | ND |
| 100060338-5 | NHC-TP | 2.0E−04M | 205 nm | — | — | — | — | ND |

Aqueous solubility (simulated intestinal fluid)

| | | | | | | | | |
|---|---|---|---|---|---|---|---|---|
| 100060338-1 | Example 1 | 2.0E−04M | 230 nm | 13.87 | 18.29 | 16.1 | 100 | |
| 100060338-2 | NHC | 2.0E−04M | 230 nm | 244.66 | 263.52 | 200 | 100 | ADJ |
| 100060338-3 | molnupiravir | 2.0E−04M | 230 nm | 301.69 | 328.92 | 200 | 100 | ADJ |
| 100060338-4 | NHC-DP | 2.0E−04M | 205 nm | — | — | — | — | ND |
| 100060338-5 | NHC-TP | 2.0E−04M | 205 nm | — | — | — | — | ND |

Aqueous solubility (simulated gastric fluid)

| | | | | | | | | |
|---|---|---|---|---|---|---|---|---|
| 100060338-1 | Example 1 | 2.0E−04M | 230 nm | <1 | <1 | <1 | 100 | BQA |
| 100060338-2 | NHC | 2.0E−04M | 230 nm | 275.19 | 210.58 | 200 | 100 | ADJ |
| 100060338-3 | molnupiravir | 2.0E−04M | 230 nm | 257.79 | 292.76 | 200 | 100 | ADJ |
| 100060338-4 | NHC-DP | 2.0E−04M | 205 nm | — | — | — | — | ND |
| 100060338-5 | NHC-TP | 2.0E−04M | 205 nm | — | — | — | — | ND |

ADJ: Adjusted. When the observed mean solubility is greater than 200 μM, the mean value is adjusted to the maximum assay concentration,
which is 200 μM. ND: Not Detected. BQA: Below the Limit of Quantitation.

| | | | | |
|---|---|---|---|---|
| Compound | Client Cpd. | Test | | |
| I.D. | I.D. | Conc. | logD | Flag |

Partition coefficient (log D, n-octanol/PBS, pH 7.4)

| | | | | |
|---|---|---|---|---|
| 100060338-1 | Example 1 | 1.0E−04M | 0.52 | |
| 100060338-2 | NHC | 1.0E−04M | 1.12 | |
| 100060338-3 | molnupiravir | 1.0E−04M | 0.39 | |
| 100060338-4 | NHC-DP | 1.0E−04M | 0.12 | |
| 100060338-5 | NHC-TP | 1.0E−04M | — | ND |

ND: Not Detected.

| Compound | Client Cpd. | Test | Permeability (10⁻⁶ cm/s) | | | | Percent Recovery(%) | | |
|---|---|---|---|---|---|---|---|---|---|
| I.D. | I.D. | Conc. | 1^{st} | 2^{nd} | Mean | Flags | 1^{st} | 2^{nd} | Mean |

A-B permeability (Caco-2, pH 6.5/7.4)

| | | | | | | | | | |
|---|---|---|---|---|---|---|---|---|---|
| 100060338-1 | Example 1 | 1.0E−05M | 36.15 | 30.68 | 33.4 | | 121 | 115 | 118 |
| 100060338-2 | NHC | 1.0E−05M | 0.55 | 0.34 | 0.4 | | 77 | 73 | 75 |
| 100060338-3 | molnupiravir | 1.0E−05M | 0.19 | 0.13 | 0.2 | | 70 | 71 | 71 |
| 100060338-4 | NHC-DP | 1.0E−05M | 0.07 | 0.06 | <0.07 | BLQ | 8 | 7 | 7 |
| 100060338-5 | NHC-TP | 1.0E−05M | — | — | — | ND | — | — | — |

B-A permeability (Caco-2, pH 6.5/7.4)

| | | | | | | | | | |
|---|---|---|---|---|---|---|---|---|---|
| 100060338-1 | Example 1 | 1.0E−05M | 26.15 | 21.17 | 23.7 | | 91 | 83 | 87 |
| 100060338-2 | NHC | 1.0E−05M | 0.27 | 0.30 | 0.3 | | 83 | 87 | 85 |
| 100060338-3 | molnupiravir | 1.0E−05M | 0.26 | 0.27 | 0.3 | | 90 | 89 | 89 |
| 100060338-4 | NHC-DP | 1.0E−05M | 0.03 | 0.02 | <0.03 | BLQ | 64 | 72 | 68 |
| 100060338-5 | NHC-TP | 1.0E−05M | — | — | — | ND | — | — | — |

BLQ: Below the Limit of Quantitation. Test compound was well detected in donor samples but not detected in receiver samples. The concentration of test compound in receiver sample was below the limit of quantitation.
ND: Not Detected. Test compound was not reliably detected in the assay matrix.

Conclusion: Although Example 1 is similar lipophilicity compared to molnupiravir and NHC according the logD, the aqueous solubility in PBS, simulated intestinal fluid and simulated gastric fluid is much worser (i.e. practically insoluble). The solubility of NHC and molnupiravir is above the measurement limit of the assay, and indeed, for molnupiravir a high solubility of 121 mM (39.7 mg/mL) in water has been mentioned in the assessment report for use of molnupiravir for the treatment of COVID-19 published by the Committee for Medicinal Products for Human Use (CHMP):

www.ema.europa.eu/en/documents/referral/lagevrio-also-known-molnupiravir-mk-4482-covid-19-article-53-procedure-assessment-report_en.pdf

For NHC a high solubility of 58 mM (15 mg/mL) in water has been mentioned upon slight warming: www.cellsignal.com/products/activators-inhibitors/beta-d-n4-hydroxycytidine/81178 Nevertheless, the permeability of zwitterionic Example 1 is improved by about two orders of magnitude compared to molnupiravir, NHC and NHC-DP, allowing a beneficial biopharmaceutics classification of Class Il (low solubility, high permeability).

### Example 4: Mouse pharmacokinetics

The present study aimed at the determination of pharmacokinetic parameters of Example 2 and it's metabolites (NHC, NHC-MP, NHC-DP and NHC-TP) in blood after single dose oral administration of 96.5 μmol/kg to female C57BL/6 mice (body weight 20 to 21 g). Blood samples were obtained from the retrobulbar venous plexus into Li-Heparin tubes (Minivette POCT, SARSTEDT). The vehicle for preparation of the dose formulations was PBS. The samples were analysed by high resolution LC-MS at Pharmacelsus GmbH, Germany.

After oral administration of 96.5 μmol/kg Example 2 the test item was not found in all blood samples (below lower limit of quantification of 14.4 nM). The metabolite NHC-DP reached a maximal blood concentration of 923+138 nM 4 h post dose and it's metabolite NHC-TP reached a maximal blood concentration of 611+100 nM 4 h post dose. The elimination half-life and the AUC (0-inf) were not calculable in both metabolites. Furthermore it's metabolite NHC-MP reached a maximal blood concentration of 274+41.4 nM 30 min post dose. The elimination half-life was 7.3 h and the AUC (-inf) was 1624 nM*h. The metabolite NHC reached a maximal blood concentration of 4239+902 nM 30 min post dose. The elimination half-life was 1.7 h and the AUC (0-inf) was 5761 nM*h.

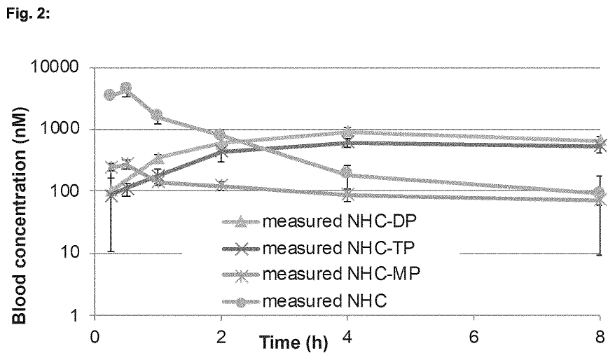

Conclusion: FIG. 2 shows the blood concentrations of NHC, NHC-PM, NHC-DP and NHC-TP, respectively, over the time period tested. Although no prodrug of dosed Example 2 is detectable, sustained concentrations of phosphorylated NHC analogs are present over a long period of time, underscoring that the rate-limiting step can be successfully bypassed with Example 2.

### Example 50

Step 1:2-Fluoro-4-(4,4,5,5-tetramethyl-1,3,2-dioxaborolan-2-yl) aniline (50a)

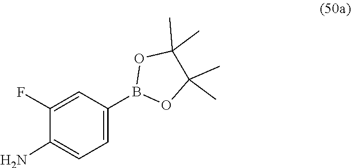

To a solution of 4-bromo-2-fluoroaniline (4.00 g, 21.1 mmol) in 1,4-dioxane (30 mL) was added bis (pinacolato) diboron (5.38 g, 21.2 mmol), KOAc (6.23 g, 63.5 mmol) and Pd (dppf) Cl2 (776 mg, 1.1 mmol). Then the mixture was heated at 90° C. for 1 h, cooled to rt, filtered, concentrated and purified by FCC(PE:EA=8:1) to give compound 50a as a white solid.

Step 2:3-Fluoro-3′-(methoxy-d3)-[1,1′-biphenyl]-4-amine (50b)

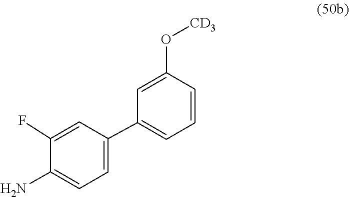 [SMILES: [2H]C([2H])([2H])Oc1cccc(-c2ccc(N)c(F)c2)c1]

To a solution of compound 50a (800 mg, 3.37 mmol) in 1,4-dioxane (10 mL) and H₂O (1 mL) was added 1-bromo-3-(methoxy-d3) benzene (638 mg, 3.36 mmol), Na₂CO₃ (1.07 g, 10.1 mmol) and Pd (dppf) Cl2 (124 mg, 0.17 mmol) and then the mixture was heated at 90° C. for 2 h, cooled to rt, filtered, concentrated and purified by FCC (PE:EA=10:1) to give compound 50b as an oil.

Step 3:2-((3-Fluoro-3′-(methoxy-d3)-[1,1′-biphenyl]-4-yl) carbamoyl) cyclopent-1-ene-1-carboxylic acid (50)

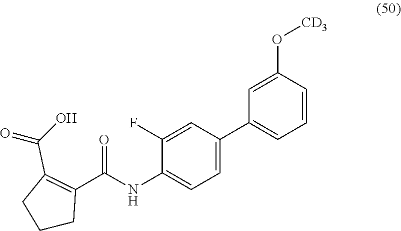 [SMILES: [2H]C([2H])([2H])Oc1cccc(-c2ccc(NC(=O)C3=C(C(=O)O)CCC3)c(F)c2)c1]

A solution of compound 50b (120 mg, 545 μmol) and 1-cyclopentene-1,2-dicarboxylic anhydride (74 mg, 540 μmol) in DCM (2.5 mL) was heated at 40° C. for 4 h. The mixture was filtered and the filter cake washed with MeCN (2×2 mL). The solid was dried in vacuum to afford compound 50 as a pale yellow solid. ¹H-NMR (400 MHZ, DMSO-d6) δ13.04 (br s, 1H), 10.58 (s, 1H), 8.07 (t, J=8.4 Hz, 1H), 7.63 (d, J=12.4 Hz, 1H), 7.53 (d, J=8.4 Hz, 1H), 7.37 (t, J=8.0 Hz, 1H), 7.27-7.23 (m, 2H), 6.94 (dd, J=8.0, 2.0 Hz, 1H), 2.80 (br s, 2H), 2.69 (br s, 2H), 1.93-1.85 (m, 2H). LCMS (ESI): m/z 359.0 (M+H)*.

### Example 51

Step 1:2,6-Difluoro-4-(4,4,5,5-tetramethyl-1,3,2-dioxaborolan-2-yl) aniline (51a)

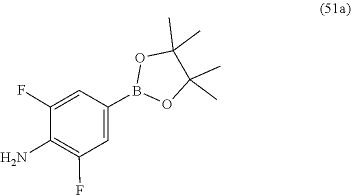 [SMILES: CC1(C)OB(c2cc(F)c(N)c(F)c2)OC1(C)C]

To a solution of 4-bromo-2,6-difluoroaniline (10 g, 48 mmol) in 1,4-dioxane (100 mL) was added bis (pinacolato) diboron (12.8 g, 50.4 mmol), CH3COOK (14.1 g, 144 mmol) and Pd (dppf) Cl2 (1.0 g, 2.40 mmol). The mixture was stirred at 90° C. under N₂ for 2 h, cooled to rt, concentrated and purified by FCC (PE:EA=10:1) to give compound 51a as a yellow solid. Step 2:3,5-Difluoro-3′-(methoxy-d3)-[1,1′-biphenyl]-4-amine (51b)

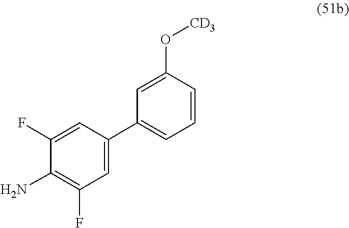 [SMILES: [2H]C([2H])([2H])Oc1cccc(-c2cc(F)c(N)c(F)c2)c1]

To a solution of compound 51a (4.5 g, 13.3 mmol) in 1,4-dioxane (50 mL) and H₂O (5 mL) was added 1-bromo-3-(methoxy-d3) benzene (3.34 g, 13.3 mmol), Na₂CO₃ (5.61 g, 39.4 mmol) and Pd (dppf) Cl2 (400 mg, 0.67 mmol). The mixture was stirred at 90° C. under N₂ for 2 h, cooled to rt, concentrated and purified by FCC (PE:EA=10:1) to give compound 51b as a yellow solid. LCMS (ESI): m/z 239.1 (M+H)*.

Step 3:2-((3,5-Difluoro-3′-(methoxy-d3)-[1,1′-biphenyl]-4-yl) carbamoyl) cyclopent-1-ene-1-carboxylic acid (51)

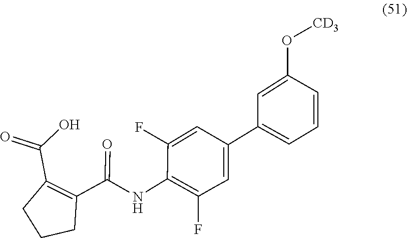 [SMILES: [2H]C([2H])([2H])Oc1cccc(C2=CCC(F)=C(NC(=O)C3=C(C(=O)O)CCC3)C(F)=C2)c1]

To a solution of compound 51b (3.40 g, 14.3 mmol) in DCM (20 mL) were added 1-cyclopentene-1,2-dicarboxylic anhydride (1.90 g, 14.3 mmol) and then the mixture was stirred at rt for 2 h. The mixture was filtered and the filter cake washed with MeCN. The solid was dried in vacuum to afford compound 51 as a white solid. 1H-NMR (500 MHZ, DMSO-d6) δ12.95 (br s, 1H), 10.13 (s, 1H), 7.55 (d, J=8.0 Hz, 2H), 7.39 (t, J=7.8 Hz, 1H), 7.32-2.83 (m, 2H), 6.99 (dd, J=1.8, 8.3 Hz, 1H), 2.81-2.79 (m, 2H), 2.69-2.66 (m, 2H), 1.97-1.89 (m, 2H). LCMS (ESI): m/z 377.3 (M+H)*.

### Example 99: Human DHODH Inhibition Assay

The in vitro inhibition of hDHODH was measured using an N-terminally truncated recombinant hDHODH enzyme as described in J. Med. Chem. 2006; 49:1239. Briefly, the hDHODH concentration was adjusted in a way that an average slope of approximately 0.2 AU/min served as the positive control (e.g. without inhibitor). The standard assay mixture contained 60 UM 2,6-dichloroindophenol, 50 μM decylubiquinone and 100 UM dihydroorotate. The hDHODH enzyme with or without at least six different concentrations of the compounds was added and measurements were performed in 50 mM TrisHCI, 150 mM KCl and 0.1% Triton X-100 at pH 8.0 and at 30° C. The reaction was started by adding dihydroorotate and measuring the absorption at 600 nm for 2 min. For the determination of the IC₅₀ values, each data point was recorded in triplicate. For the determination of the inhibitory constant Kᵢ, the K_{M} values for DHO and decylubichinon were determined. Afterwards, the compounds were diluted in a dilution series depending on their IC₅₀ values in DMSO. The dilution was: 0 ×IC₅₀ , 1/4×IC₅₀ , 1/2×IC₅₀ , 1×IC₅₀ , 2×IC₅₀ , 4×IC₅₀ . In addition, the substrate concentration for DHO and decylubichinon were varied 1/4×KM, 1/2×KM, 1×KM, 2×KM, 4×KM in further dilution series with separate measurement of DHO and decylubiquinone. Each data point was recorded in duplicate.

The Kᵢ values for examples of the present invention were in the range of the non-deuterated matched pair (Example C26 from WO2003/006425):

(C26)

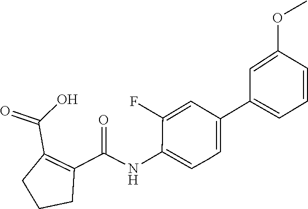 [SMILES: COc1cccc(C2=CCC=C(NC(=O)C3=C(C(=O)O)CCC3)C(F)=C2)c1]

| | Kᵢ (DHO) | Kᵢ (decylubichinon) |
|---|---|---|
| Example # | [nM] | [nM] |
| C26 | 592 | 245 |
| 50 | 521 | 234 |

As shown above, the DHODH inhibition of deuterated analog 50 compared to the non-deuterated matched pair (Example C26 from WO2003/006425) is not affected, however the microsomal stability is improved.

### Example 100: Antiviral Activity on SARS-COV-2

The viral replication (YFP) and cell viability assay has been described in Pathogens 2021; 10:1076 and furnished the following results:

| Example # | EC₅₀ | CC₅₀ | SI |
|---|---|---|---|
| C1 | >50 μM | >100 μM | inactive |
| 1 | <10 μM | >100 μM | >10 |
| 50 | <10 μM | >100 μM | >10 |
| 51 | <20 μM | >100 μM | >5 |

Contrary to citicoline (C₁), its analog 1 showed antiviral activity against SARS-COV-2. In addition, the DHODH inhibitors 50 and 51 show similar antiviral activity.

### Example 101: Synergistic Antiviral Activity on SARS-COV-2 with an DHODH Inhibitor

The synergistic potential of compound 1 together with the DHODH inhibitor 50

The method of combinatorial drug assessment by a viral replication inhibition assay has been published in Pathogens 2021; 10:1076. Caco-2 cells were cultivated in 96-well plates at 25000 cells/well, infected with SARS-COV-2 d6-YFP at an MOI of 0.003 and treated with Example 50, Example 1 or a combination of the drugs, starting at the respective 4×EC50 concentrations of the single compounds. Viral replication was determined as 30 h post infection (p.i.) by quantitative fluorescence detection of virus-driven YFP expression in the fixed cells. Inhibitory profiles of viral replication measured through virus-encoded YFP reporter expression are presented in a bar chart of quadruplicate determinations (mean+SD). The combinatorial drug assessment was calculated by using the CompuSyn algorithm as described in Int. J. Mol. Sci. 2021; 22:575.

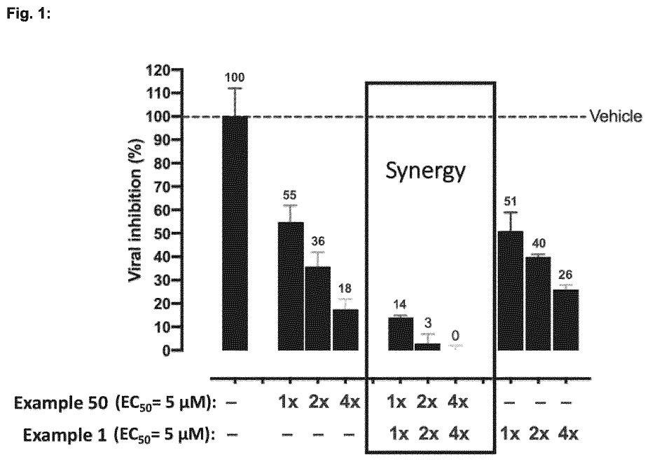

A representative experiment is shown in FIG. 1. Compound 1 shows synergistic antiviral effects on SARS-COV-2 when combined with an DHODH inhibitor of Example 50.

### Example 102: Antiviral Activity on HRV-14, Influenza a and RSV

In collaboration with the National Institute of Health (NIH), the antiviral activity of the nucleoside analog Example 1 was tested against human rhinovirus-14 (HRV-14), influenza A and respiratory syncytial virus (RSV) A2. Hela cells were used for HRV-14, MDCK cells for influenza A and MA-104 cells for RSV A2. Cells were treated with a serial log 10 dilution of Example 1 and subsequently infected with human rhinovirus 14 (HRV-14), influenza A (H₁N1, California/07/2009) or RSV A2. EC₅₀ concentration was determined by linear regression and CC50 concentration by neutral red. For Example 1, the following measured values were obtained:

| Virus | EC₅₀ | CC₅₀ | SI |
|---|---|---|---|
| HRV-14 | 4.7 μM | >25 μM | >5.8 |
| Influenza A | 4.8 μM | 25 μM | 5.2 |
| RSV A2 | 1.1 μM | 2.4 μM | 2.2 |

### Example 103: Antiviral Activity on HRV-14 and RSV in Combination with a DHODH Inhibitor

Furthermore, in vitro combination assays (AP2B) with DHODH inhibitor Example 50 and nucleoside analog Example 1 were performed to investigate antiviral activity against RSV and HRV-14. For both viruses, there was an improvement in antiviral activity after combining the two compounds.

RSV: MA-104 cells were treated either in monotherapy with a serial dilution of Example 50 or Example 1, or a combination of 25 UM of Example 50 with serial dilution of Example 1 and then infected with RSV A2. Virus concentrations were determined by endpoint titration (CCID50) and yielded the following results:

RSV Virus Titers - *CCID₅₀/mL (Log10)

| | | | Example 1 + 25 μM |
|---|---|---|---|
| Conc. (μM) | Example 50 | Example 1 | Example 50 |
| 25 | <0.7 | 3.5 | <0.7 |
| 7.9 | 2.0 | 3.5 | <0.7 |
| 2.5 | 2.0 | 3.5 | <0.7 |
| 0.79 | 3.5 | 4.0 | <0.7 |
| 0.25 | 3.5 | 4.0 | <0.7 |
| 0.079 | 3.5 | 3.5 | <0.7 |
| 0.025 | 3.7 | 4.0 | <0.7 |
| 0.0079 | 3.7 | 4.5 | <0.7 |

*CCID₅₀ = 50% cell culture infectious dose
4.0 (virus control)

HRV-14: Hela cells were treated either in monotherapy with a serial dilution of Example 50 or Example 1, or a combination of 25 UM of Example 50 with serial dilution of Example 1 and then infected with HRV-14. Virus concentrations were determined by endpoint titration (CCID₅₀) and yielded the following results:

HRV-14 Virus Titers - *CCID₅₀/mL (Log10)

| | | | Example 1 + 25 μM |
|---|---|---|---|
| Conc. (μM) | Example 50 | Example 1 | Example 50 |
| 25 | 3.5 | 3.5 | <0.7 |
| 7.9 | 4.0 | 4.7 | <0.7 |
| 2.5 | 7.0 | 6.0 | <0.7 |
| 0.79 | 6.7 | 6.7 | 3.7 |
| 0.25 | 6.7 | 7.0 | 3.5 |
| 0.079 | 7.0 | 6.7 | 4.0 |
| 0.025 | 6.7 | 6.7 | 3.7 |
| 0.0079 | 6.7 | 6.7 | 4.5 |

*CCID₅₀ = 50% cell culture infectious dose
6.7 (virus control)

Conclusion: Combination of a DHODH inhibitor with Example 1 showed a synergistic behavior also on human rhinovirus-14 (HRV-14) and respiratory syncytial virus (RSV).

1. A method of preventing and/or treating a disease in a mammalian subject in need thereof, comprising administering to the mammalian subject a compound according to Formula (I): 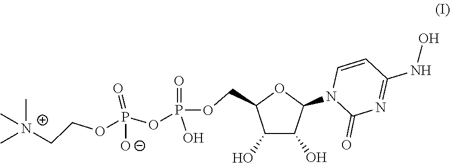 [SMILES: C[N+](C)(C)CCOP(=O)([O-])OP(=O)(O)OC[C@H]1O[C@@H](n2ccc(NO)nc2=O)[C@H](O)[C@@H]1O]
or a tautomer, solvate or pharmaceutically acceptable salt thereof.

2. The method according to claim 1, wherein the disease is a viral infection caused by an RNA virus.

3. The method according to claim 1, wherein the disease is a viral infection caused by a coronavirus.

4. The method according to claim 1, wherein the disease is selected from AIDS, hepatitis, ebola, polio, diarrhea, measles, mumps, rabies, Lassa fever, viral flu, respiratory disease, acute respiratory disease, sepsis, acute respiratory distress syndrome, long COVID, a cytokine storm, and an adverse immune reactions.

5. The method according to claim 1, wherein the compound of Formula (I) or a tautomer, solvate, or pharmaceutically-acceptable salt thereof is administered in combination with a standard antiviral therapy (SAT).

6. The method according to claim 1, wherein the compound of Formula (I) or a tautomer, solvate, or pharmaceutically-acceptable salt thereof is administered in combination with a DHODH inhibitor.

7. The method according to claim 6, wherein the DHODH inhibitor comprises at least one compound of Formula (II): 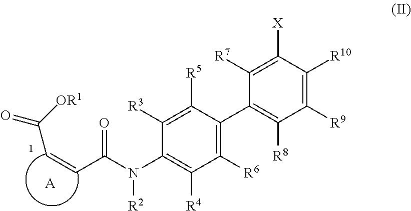
or an enantiomer, diastereomer, tautomer, prodrug, solvate, or pharmaceutically acceptable salt thereof, wherein
R¹ and R² are independently selected from H and D;
R³, R⁴, R⁵ and R⁶ are independently selected from H, D, halogen, CN, C₁₋₄-alkyl, O-C₁₋₄-alkyl, fluoro-C₁₋₄-alkyl and O-fluoro-C₁₋₄-alkyl, having one or more hydrogen atoms in alkyl optionally replaced by deuterium;
R⁷, R⁸, R⁹ and R¹⁰ are independently selected from H, D, halogen, CN, C₁₋₄-alkyl, O-C₁₋₄-alkyl, fluoro-C₁₋₄-alkyl and O-fluoro-C₁₋₄-alkyl, having one or more hydrogen atoms in alkyl optionally replaced by deuterium;
X is selected from H, D, OH, OD, S(═O)_{y}R¹¹ and OR^{11;}
R¹¹ is selected from C₁₋₄-alkyl, C₃-4-cycloalkyl and fluoro-C₁₋₄-alkyl, having one or more hydrogen atoms in alkyl optionally replaced by deuterium;
y is 0 to 2; 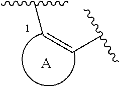
is selected from a 5-membered heteroaryl, cyclopentenyl and heterocyclopentenyl,
having one or more hydrogen atoms optionally replaced by deuterium, said A is unsubstituted or substituted with 1 to 5 substituents independently selected from the group consisting of halogen, CN, NO₂, oxo, OH, C₁₋₄-alkyl, O-C₁₋₄-alkyl, fluoro-C₁₋₄-alkyl and O-fluoro-C₁₋₄-alkyl, CO₂H and SO₃H, having one or more hydrogen atoms in alkyl optionally replaced by deuterium;
provided that at least one hydrogen in R³, R⁴, R⁵, R⁶, R⁷, R⁸, R⁹, R¹⁰, R¹¹, X and/or A is replaced by deuterium.

8. The method according to claim 7, wherein the DHODH inhibitor is selected from 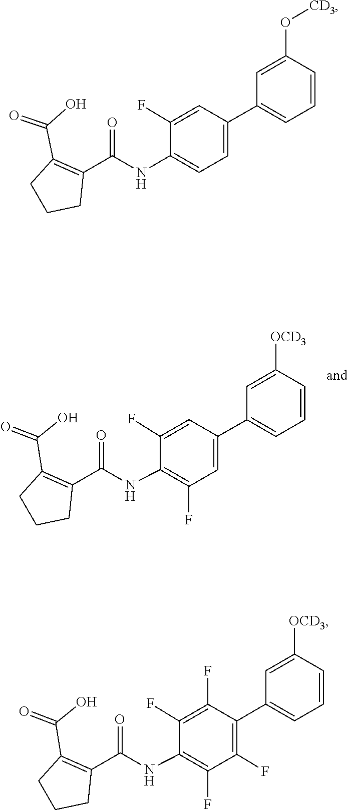
or a solvate or pharmaceutically acceptable salt thereof.

9. The method according to claim 1, wherein the compound of Formula (I) or a tautomer, solvate, or pharmaceutically-acceptable salt thereof is administered as part of a pharmaceutical composition.

10. The method according to claim 9, wherein the pharmaceutical composition further comprises one or more pharmaceutically acceptable carriers or excipients.

11. A method of preparation of a compound according to Formula (I) 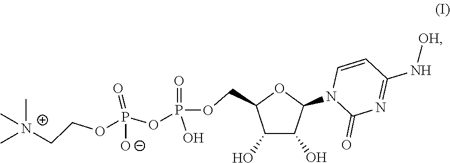 [SMILES: C[N+](C)(C)CCOP(=O)([O-])OP(=O)(O)OC[C@H]1O[C@@H](n2ccc(NO)nc2=O)[C@H](O)[C@@H]1O]
comprising reacting citicoline or salts thereof with hydroxylamine or salts thereof.

12. A method of treating a disease in a mammalian subject in need thereof, comprising administered to the mammalian subject a compound according to Formula (I): 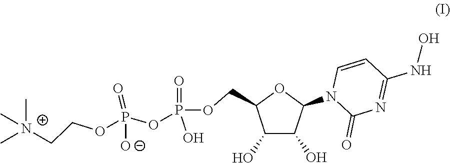 [SMILES: C[N+](C)(C)CCOP(=O)([O-])OP(=O)(O)OC[C@H]1O[C@@H](n2ccc(NO)nc2=O)[C@H](O)[C@@H]1O]
or a tautomer, solvate or pharmaceutically acceptable salt thereof, wherein the disease is selected from AIDS, hepatitis, ebola, polio, diarrhea, measles, mumps, rabies, Lassa fever, viral flu, respiratory disease, acute respiratory disease, sepsis, acute respiratory distress syndrome, long COVID, a cytokine storm, and an adverse immune reactions.

13. The method of claim 2, wherein the RNA virus is HIV, HCV, Ebola, rotavirus, Zika virus, polio virus, rhinovirus, hepatitis A virus, measles virus, mumps virus, RSV, rabies, Lassa virus, hantavirus, or influenza.

14. The method of claim 4, wherein the disease is caused by SARS-COV-2 and/or COVID-19, and wherein the disease is a moderate to severe case of the disease.

15. The method of claim 12, wherein the disease is caused by SARS-COV-2 and/or COVID-19, and wherein the disease is a moderate to severe case of the disease.

16. The method of claim 6, further comprising administering a standard antiviral therapy (SAT).

17. The method of claim 9, wherein the pharmaceutical composition is chosen from a tablet, capsule, granule, powder, sachet, reconstitutable powder, dry powder inhaler or chewable.

18. The method of claim 13, wherein the RNA virus is a single stranded RNA virus.

19. The method of claim 18, wherein the single stranded RNA virus is selected from HCOV-229E, HCoV-NL63, or a betacoronavirus.

20. The method of claim 19, wherein the betacoronavirus is selected from HCoV-OC43, SARS-COV-1, HCoV-HKU1, MERS-COV, or SARS-COV-2.
